(19) United States
(12) Patent Application Publication
(10) Pub. No.: US 2015/0157511 A1
(43) Pub. Date: Jun. 11, 2015

(54) FEMININE HYGIENE DEVICE WITH WITHDRAWAL MEMBER

(71) Applicant: The Procter & Gamble Company, Cincinnati, OH (US)

(72) Inventors: Kevin Charles Strong, Loveland, OH (US); Evan Joseph Durling, West Chester, OH (US); David Zong Pan, Cambridge, MA (US)

(73) Assignee: The Procter & Gamble Company, Cincinnati, OH (US)

(21) Appl. No.: 14/101,756

(22) Filed: Dec. 10, 2013

(51) Int. Cl. A61F 13/34

(52) U.S. Cl. CPC A61F 13/34

(57) ABSTRACT

An absorbent article comprising a withdrawal member. The withdrawal member is attached to the absorbent material such that it only crosses one point on the perimeter of the fibrous structure.

### FIELD OF THE INVENTION

This invention relates to a feminine hygiene device with a withdrawal member. More specifically, this invention relates to a tampon wherein the tampon has a withdrawal member that is attached to a portion of the length of the tampon such that the withdrawal member only crosses one point on the perimeter of the tampon pledget.

### BACKGROUND OF THE INVENTION

Tampons are a form of feminine hygiene devices inserted in the vaginal canal for the absorption of menses. Tampons may be inserted into the vaginal canal using an applicator or digitally. Many tampons have a withdrawal member attached to the tampon. The withdrawal member is traditionally attached by one of two means: sewing across the entire length of the tampon pledget in a continuous manner followed by separating the tampon pledgets or by crossing through the tampon and being tied to itself.

Sewing a withdrawal member along the entire length of the tampon pledget in a continuous manner such that it crosses two points on the pledget perimeter uses excess withdrawal member. Creating a space for the withdrawal member to cross through the tampon pledget represents a hole in absorbency. As such, it would be beneficial to create a tampon pledget wherein the withdrawal member is attached to a portion of the tampon pledget such that it only contacts one point along the perimeter of the tampon pledget without crossing through the pledget.

### SUMMARY OF THE INVENTION

An absorbent article comprising a fibrous structure which comprises a first surface, a second surface opposed to the first surface, and a perimeter; and a withdrawal member attached to the fibrous structure such that the withdrawal member crosses no more than one point along the perimeter of the fibrous structure.

An absorbent article comprising a fibrous structure which comprises a first surface, a second surface opposed to the first surface, and a perimeter comprising a insertion end opposed to a trailing end and a first longitudinal edge opposed to a second longitudinal edge; and a withdrawal member attached to the fibrous structure such that the withdrawal member crosses no more than one point along the perimeter of the fibrous structure.

### BRIEF DESCRIPTION OF THE DRAWINGS

While the specification concludes with claims particularly pointing out and distinctly claiming the subject matter that is regarded as forming the present invention, it is believed that the invention will be better understood from the following description, taken in conjunction with the accompanying drawings, in which:

FIG. 1 is a perspective view of a feminine hygiene article.

FIG. 2 is a plan view of a fibrous structure.

FIG. 3 is a plan view of an alternate shape of a fibrous structure.

FIG. 4 is a plan view of an alternate shape of a fibrous structure.

FIGS. 5A-F are plan views of a fibrous structure with a withdrawal member attached.

### DETAILED DESCRIPTION OF THE INVENTION

The present invention is directed to a feminine hygiene device. The feminine hygiene device contains a fibrous structure having a withdrawal member attached to the fibrous structure. The fibrous structure may be compressed.

As used herein, “applicator” refers to a device or implement that facilitates the insertion of a feminine hygiene device, such as, e.g., a tampon or pessary, into an external orifice of a mammal. Exemplary applicators include telescoping, insertion portion and plunger, and compact applicators.

The term “attached,” as used herein, encompasses: configurations in which a first element may be directly secured to a second element by affixing the first element directly to the second element; configurations in which the first element may be indirectly secured to the second element by affixing the first element to an intermediate member which in turn may be affixed to the second element; and configurations in which the first element may be integral with a second element, i.e., the first element may be essentially part of the second element. Attachment may be done by mechanical or chemical means.

The term “compressed,” as used herein, refers to pressing or squeezing together or otherwise manipulating the size, shape, and/or volume to obtain a generally elongated absorbent member having a vaginally insertable shape.

As used herein, the term “feminine hygiene device” includes absorbent articles useful for feminine needs, such as articles that typically can be intended for feminine use internally, such as, for example, within a user's vagina. Internal feminine hygiene devices can include, for example, tampons and pessaries.

A “fiber” as used herein, refers to any material that can be part of a fibrous structure. Fibers can be natural or synthetic. Fibers can be absorbent or non-absorbent.

A “fibrous structure” as used herein, refers to materials which can be broken into one or more fibers. A fibrous structure can be absorbent or adsorbent. A fibrous structure can exhibit capillary action.

As used here, the terms “pledget” and “tampon pledget” are intended to be interchangeable and refer to a construction of absorbent material prior to compression of such construction into a tampon. A tampon pledget is sometimes referred to as a tampon blank, or a softwind, and the term “pledget” is intended to include such terms as well. The “pledget” may also be described as a fibrous structure.

As used herein, the term “tampon” refers to any type of absorbent structure that can be inserted into the vaginal canal or other body cavity, such as, e.g., for the absorption of fluid, to aid in wound healing, and/or for the delivery of materials, such as moisture or active materials such as medicaments.

As used herein, the term “vaginal canal” refers to the internal genitalia of the human female in the pudendal region of the body. The terms “vaginal canal” or “within the vagina” as used herein are intended to refer to the space located between the introitus of the vagina (sometimes referred to as the sphincter of the vagina) and the cervix.

The term “vaginally insertable shape” as used herein, refers to the geometrical form of the fibrous structure after compression. The fibrous structure may be compressed into a generally cylindrical configuration in the radial direction along the longitudinal and/or lateral axes, axially, or in both the radial and axial directions. An example of a typical compressed fibrous structure may be one which may be about 10-16 mm wide and about 40-50 mm long depending on the level of absorbency. While the fibrous structure may be compressed into a substantially cylindrical configuration, other shapes are possible. These fibrous structures may include shapes having a cross section that may be described as rectangular, trapezoidal, semi-circular, hourglass, or other suitable shapes.

The term “volume” as used herein, refers to the volume of the fibers and the void space within the fibrous structure. Volume is measured by the multiplication of the length by the width by the thickness of the fibrous structure.

As used herein, “cm” is centimeter, “mm” is millimeter, “g” is gram, “gsm” is grams per meter squared, “dpf” is denier per fiber, “g/g” is gram of fluid per gram of sample, “wt” is weight, “psi” is pound per square inch.

While particular embodiments of the present invention have been illustrated and described, it would be obvious to those skilled in the art that various other changes and modifications can be made without departing from the spirit and scope of the invention.

The present invention is related to a feminine hygiene device comprising an absorbent material forming a fibrous structure, specifically a tampon. The fibrous structure has a perimeter defined as the continuous line that forms the outer edge of the fibrous structure. In an embodiment, the fibrous structure has an insertion end and a trailing end, opposite the insertion end. The fibrous structure can also comprise an intermediate region between the trailing end and the insertion end. The perimeter of the fibrous structure is defined by the insertion end, the trailing end, a first longitudinal edge, and a second longitudinal edge. The fibrous structure can be wrapped with an overwrap. A withdrawal member is attached to the fibrous structure such that the withdrawal member has one locus of overlap with the perimeter of the fibrous material wherein the outermost edge of the absorbent material is designated as its perimeter and when the withdrawal member's actual configuration is projected onto this perimeter as both are present in the feminine hygiene device of the invention, there is only one locus of overlap or intersection between the withdrawal member projection and the absorbent material perimeter.

Considering the fibrous structure, uncompressed state, in one embodiment, the fibrous structure may have a width from about 190 mm broken up into a central portion and two lateral portions. Each lateral portion contacts a longitudinal edge. The central portion may have a width from about 3 mm to about 30 mm. In one embodiment, each of a first lateral portion and a second lateral portion can have a width from about 5 mm to about 80 mm In one embodiment, a central portion may comprise less than about 60% of the total width of the fibrous structure. Each of the first and second lateral portions may have a thickness from about 5 mm to about 50 mm.

The withdrawal member may be non-absorbent along at least the location of attachment to the fibrous structure. As used herein, the term “non-absorbent” refers to a structure that does not retain a significant portion of deposited fluid in its structure. The entire withdrawal member may be made non-absorbent, if desired. The materials comprising the withdrawal member may be inherently non-wettable or hydrophobic, or they may be treated to provide such properties. For example, a coating of wax may be applied to the withdrawal member to decrease or eliminate its absorbency. The withdrawal member need not necessarily be non-wicking, even if a non-absorbent withdrawal member is desired.

The withdrawal member may be attached to the fibrous structure first surface or the fibrous structure second surface. The withdrawal member may be attached to an overwrap covering the fibrous structure first surface or the fibrous structure second surface. The withdrawal member does not cross through the fibrous structure first surface to the fibrous structure second surface.

The withdrawal member may be attached to the absorbent material in any suitable manner known in the art including sewing, adhesive attachment, or a combination of known bonding methods. The withdrawal member only crosses one point along the perimeter of the absorbent material or fibrous structure such that when the withdrawal member's actual configuration is projected onto the perimeter of the absorbent material there is only one locus of overlap or intersection between the withdrawal member projection and the absorbent material perimeter.

In an embodiment, the withdrawal member does not cross through the absorbent material. The point or locus along the perimeter of the absorbent material may not be wider than the width of the withdrawal member as defined in a flat state.

The withdrawal member may be attached to a portion of the absorbent material. In an embodiment, the withdrawal member is attached to the feminine hygiene device along a line parallel to the feminine hygiene device's longitudinal axis for a percentage of the longitudinal axis of the feminine hygiene device, such as, for example 90%, 80%, 70%, 60%, 50%, 40%, 30%, 20%, or 10%.

The withdrawal member may be attached to a portion of the absorbent material. In an embodiment, the withdrawal member bisects the feminine hygiene device along its longitudinal axis for a percentage of the longitudinal axis of the feminine hygiene device, such as, for example 90%, 80%, 70%, 60%, 50%, 40%, 30%, 20%, or 10%.

The withdrawal member may be attached to a portion of the absorbent material. In an embodiment, the withdrawal member is attached to the feminine hygiene device along a parallel line to the feminine hygiene device's lateral axis for a percentage of the lateral axis of the feminine hygiene device, such as, for example 90%, 80%, 70%, 60%, 50%, 40%, 30%, 20%, or 10%.

In an embodiment, the withdrawal member bisects the feminine hygiene device along its lateral axis for a percentage of the lateral axis of the feminine hygiene device, such as, for example 90%, 80%, 70%, 60%, 50%, 40%, 30%, 20%, or 10%.

In an embodiment, the withdrawal member is sewn to the absorbent material by between 1 and 20 stitches, preferably 2 and 10 stitches, such as, for example 2 stitches, 3 stitches, 4 stitches, 5 stitches, 6 stitches, 7 stitches, 8 stitches, 9 stitches, and 10 stitches. The withdrawal member may be sewn in a direction that parallels the lateral axis. The withdrawal member may be sewn in a direction that parallels the longitudinal axis. In an embodiment, the withdrawal member may be sewn in a serpentine pattern crossing either the longitudinal or lateral axis. In an embodiment, the withdrawal member may be attached in a diagonal crossing through the longitudinal or lateral axis.

In an embodiment, the withdrawal member is sewn to the absorbent article at a stitch pitch of between 1 mm and 15 mm, such as, for example 2 mm, 3 mm, 4 mm, 5 mm, 6 mm, 7 mm, 8 mm, 9 mm, 10 mm, 11 mm, 12 mm, 13 mm, or 14 mm In an embodiment, the stitch pitch is 3.2 mm.

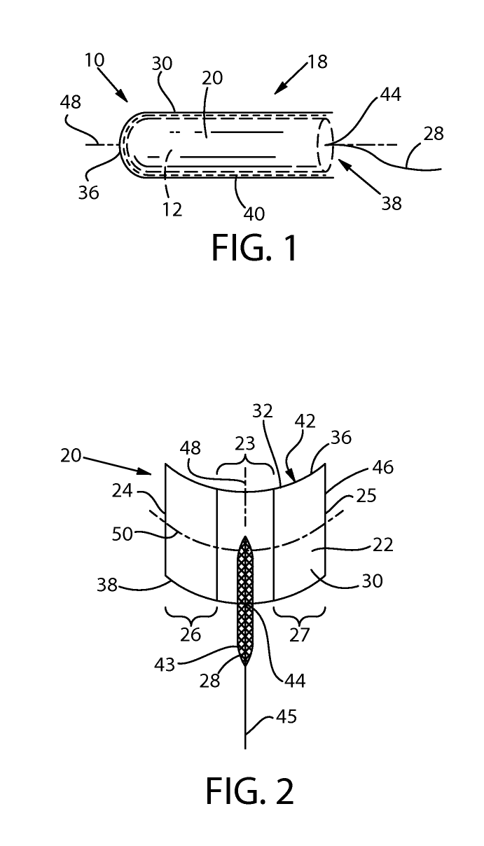

FIG. 1 exemplifies a finished feminine hygiene device 10 made from an absorbent material 18 such as a fibrous structure 20 in a vaginally insertable shape. The feminine hygiene device, however, is not limited to a structure having the particular configuration shown in the drawings. The feminine hygiene device 10 may comprise a compressed fibrous structure 12 comprising fibers 30. The compressed fibrous structure 12 can have an overwrap 40 that substantially covers the fibers 30 of the compressed fibrous structure 12. The feminine hygiene device 10 includes a withdrawal member 28 that crosses the fibrous structure at one point 44 extending beyond the trailing end 38 opposite the insertion end 36. A longitudinal axis 48 crosses through the compressed fibrous structure 12.

The fibrous structure 20 can be compressed into a tampon that can be, for example, a generally cylindrical configuration, as shown in FIG. 1. However, the fibrous structure 20 can be compressed into other shapes including, for example, a rectangular shape or a semi-circular shape. The fibrous structure can have a cross sectional shape such as a circle, rectangular, triangular, semi-circular, and other shapes that would allow for use as a tampon.

The withdrawal member 28 can be used to withdraw the feminine hygiene device 10 post use, to aid during insertion of the feminine hygiene device 10, and/or to offer reassurance of proper placement post insertion of the feminine hygiene device 10. Further, the withdrawal member 20 can signal proper placement post insertion by remaining external of the vagina.

The withdrawal member 28 is attached such that the feminine hygiene device 10 can withstand use and post use removal. In one example embodiment, the withdrawal member 28 can be sewn to the fibrous structure 20. In another example embodiment, the withdrawal member 28 can be chemically attached to the fibrous structure 20.

The withdrawal member 28 can be made from various materials to provide varying absorbency characteristics. In one embodiment, the withdrawal member 28 can be made hydrophobic to decrease or eliminate any absorbency characteristics. In another embodiment, the withdrawal member 28 can be made of absorbent materials, such as rayon and cotton. A withdrawal member 28 made from an absorbent material can have absorbency characteristics referred to as a third absorbency. In yet another embodiment, the withdrawal member 28 can be made to have both absorbent and non-absorbent characteristics. For example, the withdrawal member 28 can be made such that the portion of the withdraw member closest to the trailing end 38 of the fibrous structure can be absorbent and the portion of the withdrawal member 28 farthest from the trailing end 38 of the fibrous structure can be hydrophobic, also referred to as non-absorbent.

FIG. 2 exemplifies a fibrous structure 20 in a flat-out, uncompressed state. The fibers 30 form a fibrous structure 20. The fibrous structure 20 comprises an insertion end 36, a trailing end 38, a first longitudinal edge 24, a second longitudinal edge 25, a first lateral portion 26, and a second lateral portion 27 which form a perimeter 46. A central portion 23 is situated between the first lateral portion 26 and the second lateral portion 27. The fibrous structure has a first surface 22 and a second surface 32 opposite the first surface 22. Compression of a fibrous structure 20 may form a compressed fibrous structure 12 (as shown in FIG. 1). A longitudinal axis 48, and a latitudinal axis 50 are shown in FIG. 2.

The fibrous structure 20 may be any suitable shape, size, material, or construction for compression or formation into a feminine hygiene device 10 having a vaginally insertable shape. The fibers 30 may form a shat that can be generally square or rectangular or take on other shapes such as trapezoidal, triangular, hemispherical, chevron or hourglass shapes.

As FIG. 2 exemplifies, the fibrous structure 20 may be a batt of fibers 30 which may be a generally “chevron shaped” pad of fibers 30. While a chevron shaped fibrous structure 20 may be suitable, the edges of the chevron may be somewhat “rounded off” in order to facilitate high speed manufacturing operations. While the fibrous structure 20 shown in FIG. 2 is generally chevron shaped, other shapes such as trapezoidal, triangular, semi-circular, and rectangular shaped are also acceptable.

As shown in FIG. 2, the withdrawal member 28 may have a slub portion 43 and a non-slub portion 45. The withdrawal member 28 may be made up of a distinguishing feature and one or more lengths of plain cord. The distinguishing feature may be a slub 43. The slub 43 may be attached onto the fibrous structure 20. The non-slub portion 45 may extend beyond the fibrous structure 20.

The withdrawal member 28 may be any suitable length, such as, for example, 200 mm or less, 150 mm or less, 100 mm or less, such as for example, 90 mm, 80 mm, 70 mm, 60 mm, 50 mm, 40 mm, 30 mm, 20 mm, 10 mm, 9 mm, 8 mm, 7 mm, 6 mm, 5 mm, 4 mm, 3 mm, 2 mm, or 1 mm. The slub may be any suitable length, such as, for example, 100 mm or less, such as for example, 50 mm, 45 mm, 40 mm, 35 mm, 30 mm, 25 mm, 20 mm, 15 mm, 10 mm, 5 mm, 4 mm, 3 mm, 2 mm, or 1 mm. The slub 43 may be a percentage of the total withdrawal member length, such as, for example, 50%, 45%, 40%, 35%, 30%, 25%, 20%, 15%, 10%, or 5%.

The withdrawal member 28 crosses the perimeter 46 of the fibrous structure 20 at one point 44. The withdrawal member 28 may parallel the longitudinal axis 48, as shown in FIG. 2. The withdrawal member 28 may be attached at the first lateral portion 26, second lateral portion 27, or the central portion 23 as shown in FIG. 2.

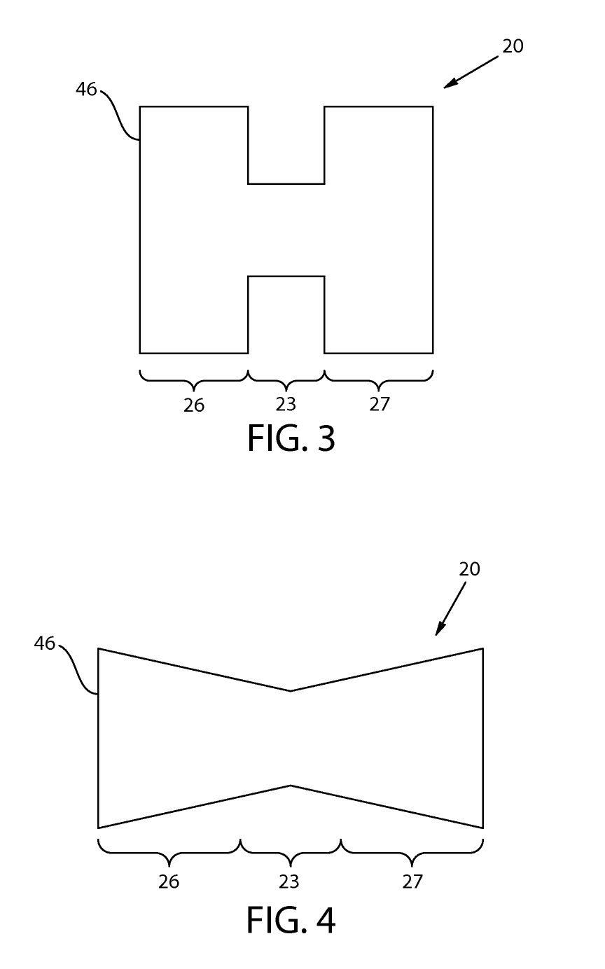

Other shapes may also be possible. For example, the fibrous structure 20 can be generally “H” shaped, such as shown in FIG. 3. A “bow tie” shaped fibrous structure 20, such as is shown in FIG. 4, is also suitable. As an alternative to the shapes of fibrous structures 20 described above, a fibrous structure 20 can have a uniform shape such as a rectangular shape, but may vary in the fiber 30 thickness along the axial extent of the fibrous structure 20.

The fibrous structure 20 may be constructed from a wide variety of liquid-absorbing materials commonly used in feminine hygiene devices such as rayon (including tri-lobal and conventional rayon fibers), cotton, or comminuted wood pulp which is generally referred to as airfelt. Examples of other suitable absorbent materials include, but are not limited to, creped cellulose wadding; meltblown polymers including coform; chemically stiffened, modified or cross-linked cellulosic fibers; synthetic fibers such as crimped polyester fibers; peat moss; foam; tissue including tissue wraps and tissue laminates; or any equivalent material or combinations of materials, or mixtures of these.

Typical fibers can comprise cotton, rayon folded tissues, woven materials, non-woven webs, synthetic and/or natural fibers or sheeting. The feminine hygiene device 10 and any component thereof, may comprise a single material or a combination of materials. Additionally, superabsorbent materials, such as superabsorbent polymers or absorbent gelling and open-celled foam materials, may be incorporated into the feminine hygiene device 10.

In another non-limiting embodiment, the fibers 30 and resulting compressed fibrous structure 12 (shown in FIG. 1) may comprise rayon, cotton, or combinations of both materials. These materials have a proven record of suitability for use in the human body. The rayon used in the fibers 30 may be any suitable type typically used in disposable feminine hygiene devices intended for in vivo use. Such acceptable types of rayon include GALAXY Rayon (a tri-lobed rayon structure) available as 6140 Rayon from Acordis Fibers Ltd., of Hollywall, England. SARILLE L rayon (a round fiber rayon), also available from Acordis Fibers Ltd. is also suitable. Any suitable cotton material may be used in the fibers 30. Suitable cotton materials include, long fiber cotton, short fiber cotton, cotton linters, T-fiber cotton, card strips, and comber cotton. The cotton may be scoured and bleached cotton absorbent with a glycerin finish, or other suitable finish.

The fibers 30 may be a laminar structure comprised of integral or discrete layers. If the compressed fibrous structure 12 of the present invention is layered, the layers may comprise different materials. For example, in one embodiment, the outer layers may comprise primarily rayon, while the intermediate layer or layers may comprise primarily cotton. Optionally, the entire compressed fibrous structure 12 may comprise a uniform or non-uniform blend of materials throughout. The absorbent material may comprise 100% rayon fibers or 100% cotton fibers. The fibers may comprise a combination of rayon and cotton fibers in any suitable combination. The fibers may comprise greater than about 25%, 30% or 40% rayon fibers and the balance of the absorbent material comprising cotton fibers. The fibers can comprise greater than about 50% rayon fibers with cotton fibers comprising the balance of the absorbent material. The absorbent material can comprise greater than about 60, 70, 75, 80 or 90% rayon fibers and the balance of the absorbent material comprising cotton fibers. In one layered embodiment, each of the layers may comprise essentially 100% of the same material, such as outer layers of 100% rayon and an intermediate layer of 100% cotton.

A typical size for fibrous structures 20 prior to compression may be from about 30 or 40 mm to about 60, 70, 80, 90 or 100 mm in length and from about 40 or 50 mm to about 70, 75, 80, 85, or 90 mm in width. The typical range for the overall basis weight may be from about 150, 200, or 250 gsm to about 600, 800, 1000 or 1100 gsm.

The feminine hygiene device can have an overwrap 40 over the fibrous structure 20. The overwrap 40 material is generally rectangular, but other shapes such as trapezoidal, triangular, hemispherical, chevron, hourglass shaped, “T” and “L” shaped are also acceptable. Optimally, the overwrap 40 may correspond to the shape of the fibrous structure 20. The overwrap 40 may be positioned around the fibrous structure 20 so that the overwrap 40 may be proximate with the insertion end 36 of the fibrous structure 20. In this regard, the overwrap 40 could exactly match up to the insertion end 36 or could, for example, extend from about 2 mm to about 8 mm over the insertion end. As well, the overwrap 40 may be proximate with the trailing end 38. In another embodiment, the overwrap 40 may extend from the trailing end 38 from about 2 mm to about 8 mm to form a skirt portion (not shown).

The overwrap 40 may substantially cover both the first surface and the second surface of the fibers 30 of the compressed fibrous structure 12. “Substantially cover” in this case means that the overwrap 40 covers at least about 50%, optionally at least about 75% or even 90% of the combined surface area of the first surface and the second surface. Thus, for example, the overwrap 40 “substantially covers” the first surface and the second surface of the absorbent material 30 when it covers 100% of the first surface and 50% of the second surface. As well, two separate pieces of overwrap 40 can form a laminate and may cover the fibers 30.

The overwrap may be attached to the fibers in at least one region of attachment. The at least one region of attachment may be less than the total surface area of the fibers. The at least one region of attachment can comprise less than about 75% of the total surface area of the fibers. In another embodiment, the at least one region of attachment may comprise less than about 50% of the total surface area of the fibers. The at least one region of attachment can comprise less than about 40% of the total surface area of the fibers. The absorbent material can comprise a central portion 23 and first and second lateral portions, 26 and 27 (shown in FIG. 2). The at least one region of attachment can be located in the central portion 23, first lateral portion 26, second lateral portion 27, and combinations thereof. The overwrap can be attached in the central portion in a region of attachment in a width that is greater than about 3 mm In another embodiment, the overwrap may be attached in the central portion in a region of attachment in a width that is greater than about 5, 7, 9, 10, 12, or 20 mm. There may be more than one region of attachment located in the central portion 23, first lateral portion 26, second lateral portion 27 and combinations thereof. The overwrap can be attached to the first and second surface areas of the fibers of the fibrous structure 20 in at least one region of attachment. The at least one region of attachment can be less than the total surface area of the first and second surface areas. Overwrap extending beyond the at least one region of attachment may be unattached to the fibers. Attachment may be accomplished by adhesives, heat bonding, pressure bonding, thermal bonding, fusion bonding, stitching, embossing, punching, or any other suitable means known in the art, and combinations thereof.

The overwrap can be embossed onto the at least one region of attachment. Embossing rolls may be utilized to attach the overwrap to the fibers. The embossing rolls may be smooth or may comprise any desired pattern of at least one protuberance or recessed areas. Such protuberance may extend from the embossing rolls in either a uniform or non-uniform thickness. In one embodiment, there may be more than one pattern of protuberance or recessed area. In another embodiment in which smooth embossing rolls may be utilized, the attachment of the overwrap to the absorbent material may result in a uniform thickness in a region of attachment. In yet another embodiment in which patterned embossing rolls, or otherwise comprising at least one protuberance or recessed area, may be utilized, the attachment of the overwrap to the fibers can result in a region of attachment comprising varying thicknesses of fibrous structures. In yet another embodiment in which embossing rolls are utilized, the attachment of the overwrap to the first and second surfaces of the fibers can result in a smooth or patterned embossment, or combinations thereof, of the first and second surfaces of the fibrous structure. For example, a first surface of the embossed fibrous structure can comprise a patterned embossment in the central portion of the first surface produced by an embossing roll comprising at least one protuberance and the second surface of the embossed fibrous structure can comprise a different patterned embossment which can be in a region of attachment in both the central portion and at least one of the lateral portions of the second surface produced by an embossing roll comprising a different pattern of at least one protuberance. Alternatively, the second surface of the embossed fibrous structure just described can be smooth as produced by a smooth embossing roll.

In one embodiment in which regions of attachment may be in both the central portion and at least one lateral portion, the thickness of the fibers in the region of attachment in the central portion may be less than the thickness of the fibers in the region of attachment in the lateral portion.

Attachment of the overwrap to the fibers may provide benefits for the overall structure of the fibers. Attachment of the overwrap to the fibrous structure may produce an area of decreased thickness within the region of attachment. Portions of the fibers outside the regions of attachment may provide areas of higher thickness fibrous structure. A benefit of decreased thickness that may be realized is an ease of attaching a withdrawal member to the fibrous structure. Attachment of a withdrawal member by a method such as stitching may benefit from a decrease in the thickness of the fibrous structure. A decrease in thickness of the absorbent material may provide for a lower loft of the fibrous structure thus allowing for an ease with which a stitching mechanism, such as a needle, may pass through the fibrous structure.

The overwrap may comprise a fibrous liquid permeable non-woven material comprising a blend of synthetic and natural fibers. The synthetic fibers may include, but are not limited to, fibers such as polyester, polyolefin, nylon, polypropylene, polyethylene, polyacrylic, cellulose acetate or bicomponent fibers. Natural fibers may include, but are not limited to, those commonly known to be non-synthetic and of natural origin such as cotton and/or rayon. In general, the natural fibers may provide ready absorption and fluid wicking strength. The ratio of synthetic fibers to natural fibers may fall in the range of from about 90:10 to about 30:70. Alternatively, the ratio of synthetic fibers to natural fibers may fall in the range of from about 70:30 to about 40:60. The synthetic fibers may have hydrophobic and/or hydrophilic surfaces. The synthetic fibers may be inherently hydrophilic, or may preferably be treated to provide such properties.

The blend of fibers forming the overwrap can be made by any number of techniques. The blends may be carded on webs. Commonly, carded webs that are hydroentangled, thermally bonded, and resin bonded all have application. In the latter case, the resin bonding agent can be used in place of the synthetic fibers as the method for tempering the aggressiveness of the natural fiber matrix. In this case, all natural fiber may be used with a significant amount of synthetic binder (10-30% by weight is common). Spunbond and meltblown processes, combining synthetic fibers extruded/spun onto/into a mat or carded web of natural fibers provide other acceptable techniques. The basis weight of the overwrap may fall into a range from about 10, 12, or 15 grams per square meter to about 30, 40, 50, or 60 grams per square meter.

The fibrous structure can have a second overwrap. The second overwrap can be a fibrous liquid permeable non-woven material comprising a blend of synthetic and natural fibers. The synthetic fibers may include, but are not limited to, fibers such as polyester, polyolefin, nylon, polypropylene, polyethylene, polyacrylic, cellulose acetate or bicomponent fibers. Natural fibers may include, but are not limited to, those commonly known to be non-synthetic and of natural origin such as cotton and/or rayon. In general, the natural fibers may provide ready absorption and fluid wicking strength. The ratio of synthetic fibers to natural fibers may fall in the range of from about 90:10 to about 30:70. Alternatively, the ratio of synthetic fibers to natural fibers may fall in the range of from about 70:30 to about 40:60. The synthetic fibers may have hydrophobic and/or hydrophilic surfaces. The synthetic fibers may be inherently hydrophilic, or may preferably be treated to provide such properties.

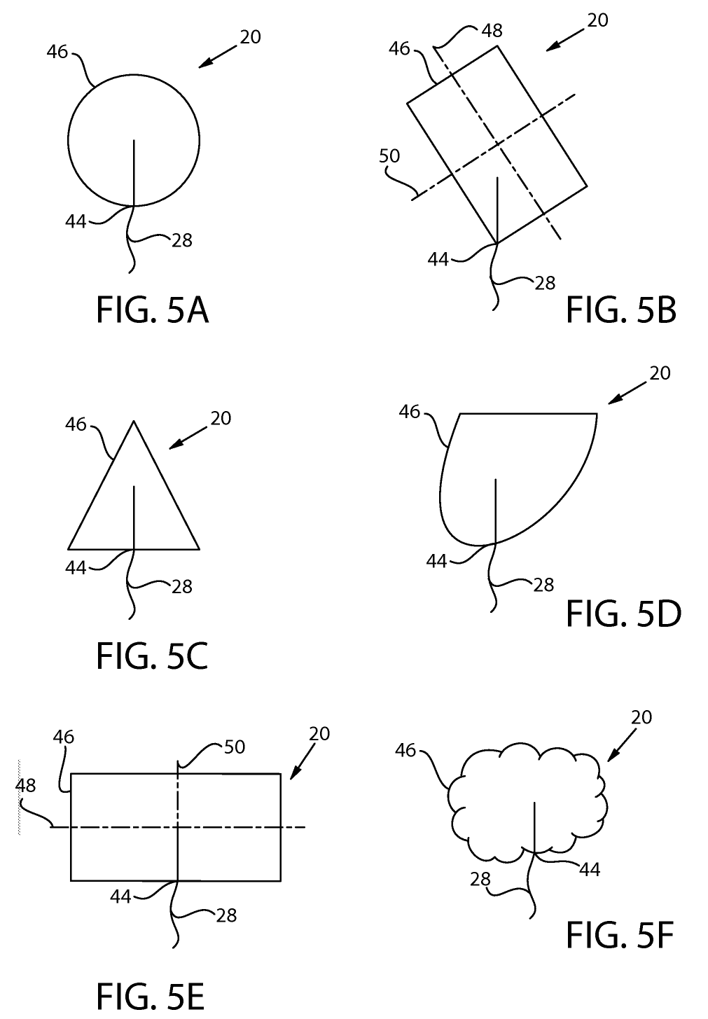

As exemplified in FIGS. 5A-F, a withdrawal member 28 may be attached to the fibrous structure such that the withdrawal member 28 crosses one point 44 along the perimeter 46 of the fibrous structure 20. As shown in FIGS. 5A-F, the fibrous structure 20 may have a cross sectional shape selected from the group consisting of a circle, a rectangular shape, a triangular shape, a semi-circular shape, and any other shapes that would allow for use as a tampon. The withdrawal member 28 may extend beyond the fibrous structure 20. As shown in the FIG. 5E, the withdrawal member 28 may be attached such that it parallels an axis, such as the longitudinal 48 or latitudinal axis 50 of the fibrous structure 20. Alternatively, the withdrawal member may be attached in a serpentine pattern to the fibrous structure. Alternatively, as shown in FIG. 5B, the withdrawal member may be attached at an angle from the longitudinal axis 48 or latitudinal axis 50. As shown in FIGS. 5B and D-E, the withdrawal member may not be attached to more than 50% of the length of any one axis of the fibrous structure. Any of the withdrawal members 28 currently known in the art may be used as a suitable withdrawal member, such as, for example, a cord. In addition, the withdrawal member 28 can take on other forms such as a ribbon, a loop, a tab, or the like. As shown in FIG. 5F, the withdrawal member 28 may be attached to a fibrous structure 20 having any nebulous shape that has a perimeter 46 which may be suitable for use as a feminine hygiene article before or after compression.

The feminine hygiene device 10 of the present invention may be inserted digitally or through the use of an applicator. Any of the currently available applicators may also be used for insertion of the feminine hygiene device 10 of the present invention.

Such applicators are typically a “tube and plunger” type arrangement and may be plastic, paper, or other suitable material. Additionally, a “compact” type applicator is also suitable.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Values disclosed herein as ends of ranges are not to be understood as being strictly limited to the exact numerical values recited. Instead, unless otherwise specified, each numerical range is intended to mean both the recited values and any integers within the range. For example a range disclosed as “1 to 10” is intended to mean “1, 2, 3, 4, 5, 6, 7, 8, 9, and 10.”

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising:
a fibrous structure which comprises a first surface, a second surface opposed to the first surface, a perimeter; and
a withdrawal member attached to the fibrous structure such that the withdrawal member crosses no more than one point along the perimeter of the fibrous structure.

2. The absorbent article of claim 1, wherein the withdrawal member is attached to the fibrous structure by 10 or less stitches.

3. The absorbent article of claim 2, wherein the withdrawal member is attached to the fibrous structure by stitches comprising a stitch pitch between 1 mm and 15 mm.

4. The absorbent article of claim 1, wherein the withdrawal member comprises an attached length that is between 5% and 80% of the longitudinal length of the fibrous structure.

5. The absorbent article of claim 1, wherein the withdrawal member comprises Rayon, cotton, comminuted wood pulp, creped cellulose wadding; meltblown polymers including coform, chemically stiffened, modified or cross-linked cellulosic fibers, synthetic fibers, or any combinations thereof.

6. The absorbent article of claim 1, wherein the withdrawal member comprises of a single cord.

7. The absorbent article of claim 1, wherein the absorbent article comprises an overwrap.

8. The absorbent article of claim 7, wherein the withdrawal member is attached to the overwrap.

9. The absorbent article of claim 1, wherein the fibrous structure has a cross sectional shape selected from the group consisting of a circle, a rectangular shape, a triangular shape, a semi-circular shape, and any other shapes that would allow for use as a tampon.

10. An absorbent article comprising:
a fibrous structure which comprises a first surface, a second surface opposed to the first surface, and a perimeter comprising a insertion end opposed to a trailing end and a first longitudinal edge opposed to a second longitudinal edge; and
a withdrawal member comprising a slub section and a non-slub section;
wherein the withdrawal member is attached to the fibrous structure such that the withdrawal member crosses no more than one point along the perimeter of the fibrous structure.

11. The absorbent article of claim 10, wherein the withdrawal member is attached to the fibrous structure by 10 or less stitches.

12. The absorbent article of claim 11, wherein the withdrawal member is attached to the fibrous structure by stitches comprising a stitch pitch between 1 mm and 15 mm.

13. The absorbent article of claim 10, wherein the withdrawal member comprises an attached length that is between 5% and 80% of the longitudinal length of the fibrous structure.

14. The absorbent article of claim 10, wherein the withdrawal member comprises Rayon, cotton, comminuted wood pulp, creped cellulose wadding; meltblown polymers including coform, chemically stiffened, modified or cross-linked cellulosic fibers, synthetic fibers, or any combinations thereof.

15. The absorbent article of claim 10, wherein the withdrawal member comprises of a single cord.

16. The absorbent article of claim 10, wherein the absorbent article comprises an overwrap.

17. The absorbent article of claim 16, wherein the withdrawal member is attached to the overwrap.

18. The absorbent article of claim 10, wherein the fibrous structure has a cross sectional shape selected from the group consisting of a circle, a rectangular shape, a triangular shape, a semi-circular shape, and any other shapes that would allow for use as a tampon.
